(19) United States
(12) Patent Application Publication
(10) Pub. No.: US 2015/0290176 A1
(43) Pub. Date: Oct. 15, 2015

(54) USE OF MTOR INHIBITORS TO TREAT VASCULAR COGNITIVE IMPAIRMENT

(71) Applicant: THE BOARD OF REGENTS OF THE UNIVERSITY OF TEXAS SYSTEM, Austin, TX (US)

(72) Inventors: Arlan Richardson, San Antonio, TX (US); Veronica Galvan, San Antonio, TX (US); Ai-Ling Lin, San Antonio, TX (US); Peter Fox, San Antonio, TX (US); Dana M. Vaughn, Seguin, TX (US)

(21) Appl. No.: 14/435,306

(22) PCT Filed: Oct. 11, 2013
(86) PCT No.: PCT/US13/64575

§ 371 Date: Apr. 13, 2015

Related U.S. Application Data
(60) Provisional application No. 61/713,407, filed on Oct. 12, 2012.

(51) Int. Cl. A61K 31/436; A61K 45/06

(52) U.S. Cl. CPC A61K 31/436; A61K 45/06

(57) ABSTRACT

Disclosed are methods and compositions for the treatment or prevention of vascular cognitive impairment. The disclosed methods and compositions include rapamycin, a rapamycin analog, or another such inhibitor of the target of rapamycin (TOR).

### CROSS REFERENCE TO RELATED APPLICATIONS

This application claims the benefit of priority of U.S. Provisional Patent Application No. 61/713,407 to Arlan Richardson et al., filed on Oct. 12, 2012, which is hereby incorporated by reference in its entirety.

### GOVERNMENT LICENSE RIGHTS

This invention was made with government support under agreement number RC2AG036613 awarded by the National Institutes of Health (NIH). The government has certain rights in the invention.

### DESCRIPTION

### Background of the Invention

A. Field of the Invention

The invention relates to methods and compositions for treating vascular cognitive impairment. The methods and compositions include rapamycin, rapamycin analogs, or other inhibitors of the mammalian target of rapamycin (“mTOR” or “mTORC1”).

B. Description of Related Art

Dementia or cognitive impairment refers to a set of symptoms that occur due to an underlying condition or disorder that causes loss of brain function. Dementia or cognitive impairment symptoms include difficulty with language, memory, perception, emotional behavior, personality (including changes in personality), or cognitive skills (including calculation, abstract thinking, problem-solving, judgment, and executive functioning skills). Dementia or cognitive impairment may be caused by a variety of underlying disorders, including Alzheimer's disease (AD), Parkinson's disease, Down's syndrome, vascular pathology (which causes vascular cognitive impairment), Lewy Body disease (which causes Lewy Body dementia), and Pick's disease (which causes Frontotemporal dementia).

The major causes of dementia or cognitive impairment are Alzheimer's disease, Lewy Body disease, and vascular pathology. Vascular pathology is believed to account for 20-30% of dementia cases, and because vascular cognitive impairment is likely underdiagnosed, it may be even more common than previously thought. A common cause of vascular cognitive impairment is the occurrence of multiple small strokes (called “mini-strokes”) that affect blood vessels and nerve fibers in the brain, which ultimately promotes symptoms of dementia or vascular cognitive impairment. Thus, vascular cognitive impairment is more common in those patients who are at risk for stroke, such as elderly patients, or patients having high blood pressure, high cholesterol, high blood sugar, or an autoimmune or inflammatory disease (such as lupus or temporal arteritis).

Treatments for non-vascular cognitive impairment symptoms or for some of the underlying causes of cognitive impairment have been proposed. For example, rapamycin and related compounds have been proposed as treatments for Alzheimer's disease, memory loss, cerebral amyloid angiopathy (CAA), Lewy Body dementia, cardiovascular disease, peripheral vascular disease, multi-infarct dementia, stroke, presenile dementia, senile dementia, and general symptoms of dementias. See U.S. Pat. No. 7,276,498; U.S. Pat. No. 7,273,874; U.S. Patent Pub. 2012/0064143; U.S. Patent Pub. 2007/0142423; U.S. Patent Pub. 2003/0176455; U.S. Patent Pub. 2003/0100577; U.S. Patent Pub. 2003/0032673; and European Patent App. EP 1 709 974. However, there is no known cure for vascular cognitive impairment, and to date, the U.S. Food and Drug Administration has not approved any drug for the treatment of vascular cognitive impairment.

### SUMMARY OF THE INVENTION

The inventors have demonstrated that inhibitors of mTOR, such as rapamycin itself, are effective for treating vascular cognitive impairment (see Examples). The inventors learned that treatment with rapamycin improved the vascular pathology and also rescued cognitive defects (e.g., learning and memory) in the subject. The effects of rapamycin on vascular pathology was surprising in light of previous studies, such as studies showing that rapamycin prohibited cell growth and/or induced cell death. Thus, the inventors demonstrate that rapamycin and other inhibitors of TOR (e.g., rapamycin analogs) can be used when neovascularization or revascularization in the central or peripheral nervous system is desired. For example, rapamycin can be used to treat or prevent diseases or disorders that are caused by an underlying vascular pathology, such as vascular cognitive impairment.

In one instance, there is disclosed a method for treating vascular cognitive impairment, the method comprising administering an effective amount of a composition comprising rapamycin or an analog of rapamycin to a subject having or suspected of having vascular cognitive impairment. In another instance, there is disclosed a method for preventing vascular cognitive impairment, the method comprising administering an effective amount of a composition comprising rapamycin or an analog of rapamycin to a subject at risk for developing vascular cognitive impairment.

The subject may be a subject that has been diagnosed as having vascular cognitive impairment. In some embodiments, the subject is a mammal. In certain aspects, the subject is a human. In certain aspects, the subject is a dog or a cat. The subject may be a subject that has a medical condition such as Alzheimer's disease, high blood pressure, high blood sugar or diabetes, or an autoimmune or inflammatory disease. In some aspects, the subject is a human subject who is greater than age 50. In some aspects, the subject is a human subject who is 50 years of age or less.

In the disclosed methods, the composition comprising rapamycin or an analog of rapamycin may be delivered in any suitable manner. In a preferred embodiment, the composition comprising rapamycin or an analog of rapamycin is orally administered to the subject.

Compositions comprising rapamycin or an analog of rapamycin may include a nanoparticle construct combined with a carrier material preferably an enteric composition for purposes of minimizing degradation of the composition until it passes the pylorus to the intestines of the subject. Compositions comprising rapamycin or an analog of rapamycin may also include a hydrophilic, swellable, hydrogel forming material. Such compositions may be encased in a coating that includes a water insoluble polymer and a hydrophilic water permeable agent. In some embodiments, the water insoluble polymer is a methyl methacrylate-methacrylic acid copolymer. Compositions comprising rapamycin or an analog of rapamycin may further include a thermoplastic polymer. Examples of the thermoplastic polymer include EUDRAGIT® Acrylic Drug Delivery Polymers (Evonik Industries AG, Germany).

The disclosed compositions comprising rapamycin or an analog of rapamycin may be comprised in a food or food additive. In some embodiments, the composition comprising rapamycin or an analog of rapamycin comprises 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99% by weight of rapamycin or an analog of rapamycin. In some embodiments, the composition comprising rapamycin or an analog of rapamycin comprises 1% to 75% by weight of rapamycin or an analog of rapamycin. In some embodiments, the composition comprising rapamycin or an analog of rapamycin comprises 25% to 60% by weight of rapamycin or an analog of rapamycin. In certain aspects, the average tissue level of rapamycin or an analog of rapamycin in the subject is greater than 0.75 pg per mg of tissue after administration of a composition comprising rapamycin or an analog of rapamycin. In some embodiments, the 24-hour trough concentration levels of rapamycin or an analog of rapamycin in the subject is greater than 1 ng/ml whole blood after administration of a composition comprising rapamycin or an analog of rapamycin.

In certain embodiments, the composition comprising rapamycin or an analog of rapamycin further comprises a second active agent. Alternatively, a subject is administered a first composition comprising rapamycin or an analog of rapamycin, and is also administered a second composition comprising a second active agent. For example, the second active agent may be eNOS, a cholinesterase inhibitor, an anti-glutamate, an anti-hypertensive agent, an anti-platelet agent, an antihyperlipidemic agent, or a medication that alleviates or treats low blood pressure, cardiac arrhythmia, or diabetes. In some embodiments, the cholinesterase inhibitor is tacrine, donepezil, rivastigmine, or galantamine. In certain aspects, the anti-glutamate is memantine. Alternatively, the second active agent may be an antibody that binds to amyloid beta (Aβ) or otherwise suppresses the formation of amyloid beta plaques in Alzheimer's Disease. Examples of such antibodies include Gantenerumab and Solanezumab.

The composition comprising rapamycin or an analog of rapamycin may be administered at the same time as the composition comprising a second active agent. Alternatively, the composition comprising rapamycin or an analog of rapamycin may be administered before the composition comprising a second active agent, or the composition comprising rapamycin or an analog of rapamycin may be administered after the composition comprising a second active agent is administered. For example, the interval of time between administration of a composition comprising rapamycin or an analog of rapamycin and a composition comprising a second active agent may be 1 to 30 days, or it may be 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more, or any integer derivable therein, hours or days.

In certain aspects, the disclosed methods and compositions improve cognitive function in a subject.

Unless otherwise specified, the percent values expressed herein are weight by weight and are in relation to the total composition.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The terms “inhibiting,” “reducing,” “treating,” or any variation of these terms, includes any measurable decrease or complete inhibition to achieve a desired result. Similarly, the term “effective” means adequate to accomplish a desired, expected, or intended result.

The use of the word “a” or “an” when used in conjunction with the term “comprising” may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps in relation to the total composition.

The compositions and methods for their use can “comprise,” “consist essentially of,” or “consist of” any of the ingredients or steps disclosed throughout the specification. With respect to the transitional phase “consisting essentially of,” in one non-limiting aspect, a basic and novel characteristic of the compositions and methods is the ability of rapamycin to treat vascular cognitive impairment.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1. Improved memory and restored cerebral blood flow (CBF) in AD mice treated with rapamycin after the onset of disease. a, Spatial learning. While learning in AD mice was impaired (14, 17, 47, 48) [*, P<0.001 and P<0.01, Bonferroni's post hoc test applied to a significant effect of genotype and treatment, F(3,188)=6.04, P=0.0014, repeated measures (RM) 2-way ANOVA], performance of rapamycin-fed AD mice was indistinguishable from non-Tg littermates' and from control-fed AD mice. No significant interaction was observed between day number and genotype (P=0.96), thus genotype and treatment had the same effect at all times during training. Overall learning was effective in all groups [F(4,188)=3.36, P=0.01, RM two-way ANOVA]. b, Spatial memory is restored by rapamycin treatment. While memory in control-fed AD mice was impaired (14, 17, 47, 48) [P values as indicated, Tukey's test applied to a significant effect of genotype and treatment (P<0.0001), one-way ANOVA], memory in rapamycin-fed AD mice was indistinguishable from non-Tg groups and was significantly improved compared to control-fed AD mice (P=0.03). c-g, Rapamycin restores CBF in AD mice. c, CBF maps and regional CBF maps (e) of representative control- and rapamycin-treated non Tg and AD mice obtained by MRI. d, Decreases in CBF in AD mice are abrogated by rapamycin treatment (P as indicated, Bonferroni's test on a significant effect of genotype and treatment on CBF, F(1,16)=14.54, P=0.0015, two-way ANOVA). f and g, Decreased hippocampal (f) but not thalamic (g) CBF in AD mice is restored by rapamycin treatment (P as indicated, Bonferroni's test on a significant effect of treatment on CBF, F(1,16)=13.62, P=0.0020, two-way ANOVA). Data are means±SEM. Panels a-b, n=10-17 per group. Panels c-g, n=6 per group.

FIG. 2. Increased vascular density without changes in glucose metabolism in rapamycin-treated AD mice. a, Cerebral metabolic rate of glucose (CMR_{Glc}) maps of representative control- and rapamycin-treated non Tg and AD Tg mice obtained by positron emission tomography. b, CMR_{Glc} as standardized uptake values (SUV) for the region of interest were not different among experimental groups (F(1,20)=0.77, P=0.39 for the effect of genotype and F(1,20)=3.63, P=0.071 for the effect of treatment, two-way ANOVA). c, Magnetic resonance angiography images of brains of rapamycin-treated non Tg and AD mice. Representative regions showing loss of vasculature in control-treated AD mice and its restoration in rapamycin-treated animals are denoted by arrows. d, Decreased cerebral vessel density in control-treated AD mice is abrogated by rapamycin treatment (P as indicated, Bonferroni's post hoc test applied to a significant effect of treatment on vascular density, F(1,16)=24.47, P=0.0001, two-way ANOVA). Data are means±SEM. n=6 per group.

FIG. 3. Reduced CAA and Aβ plaques in rapamycin-treated AD mice. a-f. Reduced Aβ plaques in rapamycin-treated AD mice. a and b, Representative images of hippocampi of control- (a) and rapamycin-treated (b) mice incubated with an Aβ-specific antibody. c-d, secondary antibodies only. d, DAPI fluorescence of the field in c. e-f, Quantitative analyses of Aβ immunoreactivity (P as indicated). g and h, Reduced microhemorrhage in rapamycin-treated AD mouse brains. g, Hemosiderin deposit. h, Quantitative analyses of numbers of hemosiderin deposits (P as indicated). i-k, Reduced CAA in rapamycin-treated AD mouse brains. Representative maximum intensity projections of stacks of confocal images of control (i) and rapamycin (j) treated AD mouse brain sections reacted with Aβ-specific antibodies and with tomato lectin to illuminate brain vasculature. k, Quantitative analyses of colocalization of Aβ immunoreactivity and tomato lectin labeling brain vasculature indicate reduced Aβ deposition on vessels in rapamycin-treated AD mice (P as indicated). l, Representative immunoblot of APP immunoreactivity in brain lysates from control- and rapamycin-treated AD mice; m, Quantitative analyses of APP immunoreactivity. Significance of differences between group means was determined using two-tailed unpaired Student's t test. Data are means×SEM. n=6-8 per experimental group.

FIG. 4. Rapamycin-induced NO-dependent vasodilation in brain. a, Rapamycin-induced cortical vasodilation. In vivo imaging of cortical vasculature illuminated by FITC-Dextran (green). Arrows indicate areas of maximal vasodilatory effect 10 min after rapamycin administration (tabbed white lines). b, Quantitative analyses of changes in diameter for cortical vessels of different sizes (P as indicated, Bonferroni's test applied to a significant effect of treatment, F(1,20)=154.12, P<0.0001, two-way ANOVA). c, Quantitative analyses of changes in diameter for cortical vessels of different sizes 10 min after treatment with acetylcholine (ACh, P as indicated, Bonferroni's test applied to a significant effect of treatment, F(1,15)=2900.20, P<0.0001, two-way ANOVA). d, Rapamycin-induced vasodilation is preceded by NO release. Arrowheads indicate regions of local NO release by DAF-FM fluorescence (green) followed by dilation of rhodamine-dextran labeled vasculature (red) in vivo. e, Rapamycin-induced vasodilation requires eNOS activation. L-NAME administration abolished rapamycin-induced NO release (DAF-FM fluorescence) and dilation of cortical vasculature. f, ACh-induced vasodilation is preceded by NO release. Uniform NO release (DAF-FM fluorescence, green) preceded vasodilation induced by ACh. g, NOS activity is required for rapamycin-induced preservation of CBF. Four weeks of intermittent L-NAME administration (once every other day) abolished rapamycin-mediated preservation of CBF in AD mice (P as indicated, Tukey's test applied to a significant effect of treatment, P<0.0001, one-way ANOVA). Data are means±SEM. n=6 per experimental group.

FIG. 5. Rapamycin levels in different brain regions of AD mice chronically fed with rapamycin-supplemented chow.

### DESCRIPTION OF ILLUSTRATIVE EMBODIMENTS

Vascular cognitive impairment is a cognitive impairment that results from underlying vascular pathology. Current approaches to treating and preventing vascular cognitive impairment focus on controlling risk factors for the vascular pathologies that underlie vascular cognitive impairment, such as high blood pressure, high cholesterol, high blood sugar or diabetes, or an autoimmune or inflammatory disease. While others have proposed treatments for some types of dementia, there is no known cure for vascular cognitive impairment, and no drug has been approved by the FDA for the treatment of vascular cognitive impairment. Thus, there is a need for methods and compositions that can treat and prevent vascular cognitive impairment.

The inventors have discovered an effective treatment for vascular cognitive impairment comprising rapamycin, an analog of rapamycin, or another inhibitor of mTOR. The inventors first learned that AD mice exhibit underlying vascular pathology, which was improved by rapamycin treatment. The rapamycin treatment also improved the cognitive defects (e.g., learning and memory) that are characteristic of AD mice. Thus, the inventors demonstrated that rapamycin and other inhibitors of TOR (e.g., rapamycin analogs) can be used to treat or prevent vascular cognitive impairment.

### A. Vascular Cognitive Impairment

The term “vascular cognitive impairment” refers to various defects caused by an underlying vascular pathology, disease, disorder, or condition that affects the brain. For example, strokes, conditions that damage or block blood vessels, or disorders such as hypertension or small vessel disease may cause vascular cognitive impairment. As used herein, the term “vascular cognitive impairment” includes mild defects, such as the milder cognitive symptoms that may occur in the earliest stages in the development of dementia, as well as the more severe cognitive symptoms that characterize later stages in the development of dementia.

The various defects that may manifest as vascular cognitive impairment include mental and emotional symptoms (slowed thinking, memory problems, general forgetfulness, unusual mood changes such as depression or irritability, hallucinations, delusions, confusion, personality changes, loss of social skills, and other cognitive defects); physical symptoms (dizziness, leg or arm weakness, tremors, moving with rapid/shuffling steps, balance problems, loss of bladder or bowel control); or behavioral symptoms (slurred speech, language problems such as difficulty finding the right words for things, getting lost in familiar surroundings, laughing or crying inappropriately, difficulty planning, organizing, or following instructions, difficulty doing things that used to come easily, reduced ability to function in daily life).

B. mTOR Inhibitors and Rapamycin

Any inhibitor of mTORC1 is contemplated for inclusion in the present compositions and methods. In particular embodiments, the inhibitor of mTORC1 is rapamycin or an analog of rapamycin. Rapamycin (also known as sirolimus and marketed under the trade name Rapamune) is a known macrolide. The molecular formula of rapamycin is C₅₁H₇₉NO₁₃.

Rapamycin binds to a member of the FK binding protein (FKBP) family, FKBP 12. The rapamycin/FKBP 12 complex binds to the protein kinase mTOR to block the activity of signal transduction pathways. Because the mTOR signaling network includes multiple tumor suppressor genes, including PTEN, LKB1, TSC1, and TSC2, and multiple proto-oncogenes including PI3K, Akt, and eEF4E, mTOR signaling plays a central role in cell survival and proliferation. Binding of the rapamycin/FKBP complex to mTOR causes arrest of the cell cycle in the G1 phase (Janus et al., 2005).

mTORC1 inhibitors also include rapamycin analogs. Many rapamycin analogs are known in the art. Non-limiting examples of analogs of rapamycin include, but are not limited to, everolimus, tacrolimus, CCI-779, ABT-578, AP-23675, AP-23573, AP-23841, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, and 42-O-(2-hydroxy)ethyl rapamycin.

Other analogs of rapamycin include: rapamycin oximes (U.S. Pat. No. 5,446,048); rapamycin aminoesters (U.S. Pat. No. 5,130,307); rapamycin dialdehydes (U.S. Pat. No. 6,680,330); rapamycin 29-enols (U.S. Pat. No. 6,677,357); O-alkylated rapamycin derivatives (U.S. Pat. No. 6,440,990); water soluble rapamycin esters (U.S. Pat. No. 5,955,457); alkylated rapamycin derivatives (U.S. Pat. No. 5,922,730); rapamycin amidino carbamates (U.S. Pat. No. 5,637,590); biotin esters of rapamycin (U.S. Pat. No. 5,504,091); carbamates of rapamycin (U.S. Pat. No. 5,567,709); rapamycin hydroxyesters (U.S. Pat. No. 5,362,718); rapamycin 42-sulfonates and 42-(N-carbalkoxy)sulfamates (U.S. Pat. No. 5,346,893); rapamycin oxepane isomers (U.S. Pat. No. 5,344,833); imidazolidyl rapamycin derivatives (U.S. Pat. No. 5,310,903); rapamycin alkoxyesters (U.S. Pat. No. 5,233,036); rapamycin pyrazoles (U.S. Pat. No. 5,164,399); acyl derivatives of rapamycin (U.S. Pat. No. 4,316,885); reduction products of rapamycin (U.S. Pat. Nos. 5,102,876 and 5,138,051); rapamycin amide esters (U.S. Pat. No. 5,118,677); rapamycin fluorinated esters (U.S. Pat. No. 5,100,883); rapamycin acetals (U.S. Pat. No. 5,151,413); oxorapamycins (U.S. Pat. No. 6,399,625); and rapamycin silyl ethers (U.S. Pat. No. 5,120,842).

Other analogs of rapamycin include those described in U.S. Pat. Nos. 6,015,809; 6,004,973; 5,985,890; 5,955,457; 5,922,730; 5,912,253; 5,780,462; 5,665,772; 5,637,590; 5,567,709; 5,563,145; 5,559,122; 5,559,120; 5,559,119; 5,559,112; 5,550,133; 5,541,192; 5,541,191; 5,532,355; 5,530,121; 5,530,007; 5,525,610; 5,521,194; 5,519,031; 5,516,780; 5,508,399; 5,508,290; 5,508,286; 5,508,285; 5,504,291; 5,504,204; 5,491,231; 5,489,680; 5,489,595; 5,488,054; 5,486,524; 5,486,523; 5,486,522; 5,484,791; 5,484,790; 5,480,989; 5,480,988; 5,463,048; 5,446,048; 5,434,260; 5,411,967; 5,391,730; 5,389,639; 5,385,910; 5,385,909; 5,385,908; 5,378,836; 5,378,696; 5,373,014; 5,362,718; 5,358,944; 5,346,893; 5,344,833; 5,302,584; 5,262,424; 5,262,423; 5,260,300; 5,260,299; 5,233,036; 5,221,740; 5,221,670; 5,202,332; 5,194,447; 5,177,203; 5,169,851; 5,164,399; 5,162,333; 5,151,413; 5,138,051; 5,130,307; 5,120,842; 5,120,727; 5,120,726; 5,120,725; 5,118,678; 5,118,677; 5,100,883; 5,023,264; 5,023,263; 5,023,262; all of which are incorporated herein by reference. Additional rapamycin analogs and derivatives can be found in the following U.S. Patent Application Pub. Nos., all of which are herein specifically incorporated by reference: 20080249123, 20080188511; 20080182867; 20080091008; 20080085880; 20080069797; 20070280992; 20070225313; 20070203172; 20070203171; 20070203170; 20070203169; 20070203168; 20070142423; 20060264453; and 20040010002.

### C. Methods of Using Rapamycin Compositions

“Treatment” and “treating” refer to administration or application of a therapeutic agent to a subject or performance of a procedure or modality on a subject for the purpose of obtaining a therapeutic benefit for a disease or health-related condition. For example, the rapamycin compositions of the present invention may be administered to a subject for the purpose of treating vascular cognitive impairment in a subject.

The terms “therapeutic benefit,” “therapeutically effective,” or “effective amount” refer to the promotion or enhancement of the well-being of a subject. This includes, but is not limited to, a reduction in the frequency or severity of the signs or symptoms of a disease. For example, administering rapamycin compositions of the present reduce the signs and symptoms of vascular cognitive impairment.

“Prevention” and “preventing” are used according to their ordinary and plain meaning. In the context of a particular disease or health-related condition, those terms refer to administration or application of an agent, drug, or remedy to a subject or performance of a procedure or modality on a subject for the purpose of preventing or delaying the onset of a disease or health-related condition. For example, one embodiment includes administering the rapamycin compositions of the present invention to a subject at risk of developing vascular cognitive impairment (e.g., an elderly patient having high blood pressure) for the purpose of preventing or delaying the onset of vascular cognitive impairment.

Rapamycin compositions, as disclosed herein, may be used to treat any disease or condition for which an inhibitor of mTOR is contemplated as effective for treating or preventing the disease or condition. For example, methods of using rapamycin compositions to treat or prevent vascular cognitive impairment are disclosed. Other uses of rapamycin are also contemplated. For example, U.S. Pat. No. 5,100,899 discloses inhibition of transplant rejection by rapamycin; U.S. Pat. No. 3,993,749 discloses rapamycin antifungal properties; U.S. Pat. No. 4,885,171 discloses antitumor activity of rapamycin against lymphatic leukemia, colon and mammary cancers, melanocarcinoma and ependymoblastoma; U.S. Pat. No. 5,206,018 discloses rapamycin treatment of malignant mammary and skin carcinomas, and central nervous system neoplasms; U.S. Pat. No. 4,401,653 discloses the use of rapamycin in combination with other agents in the treatment of tumors; U.S. Pat. No. 5,078,999 discloses a method of treating systemic lupus erythematosus with rapamycin; U.S. Pat. No. 5,080,899 discloses a method of treating pulmonary inflammation with rapamycin that is useful in the symptomatic relief of diseases in which pulmonary inflammation is a component, i.e., asthma, chronic obstructive pulmonary disease, emphysema, bronchitis, and acute respiratory distress syndrome; U.S. Pat. No. 6,670,355 discloses the use of rapamycin in treating cardiovascular, cerebral vascular, or peripheral vascular disease; U.S. Pat. No. 5,561,138 discloses the use of rapamycin in treating immune related anemia; U.S. Pat. No. 5,288,711 discloses a method of preventing or treating hyperproliferative vascular disease including intimal smooth muscle cell hyperplasia, restenosis, and vascular occlusion with rapamycin; and U.S. Pat. No. 5,321,009 discloses the use of rapamycin in treating insulin dependent diabetes mellitus.

### D. Pharmaceutical Preparations

Certain methods and compositions set forth herein are directed to administration of an effective amount of a composition comprising the rapamycin compositions of the present invention.

1. Compositions

A “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (Remington's, 1990). Except insofar as any conventional carrier is incompatible with the active ingredient, its use in the therapeutic or pharmaceutical compositions is contemplated. The compositions used in the present invention may comprise different types of carriers depending on whether it is to be administered in solid, liquid or aerosol form, and whether it needs to be sterile for such routes of administration as injection.

The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions, and these are discussed in greater detail below. For human administration, preparations should meet sterility, pyrogenicity, and general safety and purity standards as required by FDA Office of Biologics standards.

The formulation of the composition may vary depending upon the route of administration. For parenteral administration in an aqueous solution, for example, the solution should be suitably buffered if necessary and the liquid diluent first rendered isotonic with sufficient saline or glucose. In this connection, sterile aqueous media that can be employed will be known to those of skill in the art in light of the present disclosure.

In addition to the compounds formulated for parenteral administration, such as intravenous or intramuscular injection, other pharmaceutically acceptable forms include, e.g., tablets or other solids for oral administration or non-parenteral administration preferably an enteric coating formulation. Additional forms include liposomal and nanoparticle formulations; time release capsules; formulations for administration via an implantable drug delivery device, and any other form. Preferred embodiments of such nanoparticle formulations may be produced by using an anti-solvent precipitation method with an active pharmaceutical ingredient (API) to produce a heterogeneous suspension of the API loaded nanoparticle. Stability of these nanoparticles in solution may be enhanced with the addition of ionic surfactants that may promote the suspension and availability of the nanoparticles. The nanoparticles may be combined with a controlled released matrix for an effective delivery of the API via an enteral pathway. One may also use nasal solutions or sprays, aerosols or inhalants in the present invention.

The capsules may be, for example, hard shell capsules or soft-shell capsules. The capsules may optionally include one or more additional components that provide for sustained release.

In certain embodiments, the pharmaceutical composition includes at least about 0.1% by weight of the active compound. In some embodiments, the pharmaceutical composition includes at least about 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99% by weight of the active compound. In other embodiments, the pharmaceutical composition includes between about 1% to about 75% of the weight of the composition, between about 2% to about 75% of the weight of the composition, or between about 25% to about 60% by weight of the composition, for example, and any range derivable therein.

The compositions may comprise various antioxidants to retard oxidation of one or more components. Additionally, the prevention of the action of microorganisms can be accomplished by preservatives such as various antibacterial and antifungal agents, including but not limited to parabens (e.g., methylparabens, propylparabens), chlorobutanol, phenol, sorbic acid, thimerosal or combinations thereof. The composition should be stable under the conditions of manufacture and storage, and preserved against the contaminating action of microorganisms, such as bacteria and fungi.

In certain preferred embodiments, an oral composition may comprise one or more binders, excipients, disintegration agents, lubricants, flavoring agents, and combinations thereof. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar, EUDRAGIT® Acrylic Drug Delivery Polymers, or any combination thereof.

In particular embodiments, prolonged absorption can be brought about by the use in the compositions of agents delaying absorption, such as, for example, aluminum mono stearate, gelatin, EUDRAGIT® Acrylic Drug Delivery Polymers or combinations thereof.

2. Routes of Administration

Upon formulation, solutions will be administered in a manner compatible with the dosage formulation and in such amount as is therapeutically effective.

The composition can be administered to the subject using any method known to those of ordinary skill in the art. For example, a pharmaceutically effective amount of the composition may be administered intravenously, intracerebrally, intracranially, intrathecally, into the substantia nigra or the region of the substantia nigra, intradermally, intraarterially,

intralesionally, intratracheally, intranasally, topically, intramuscularly, intraperitoneally, subcutaneously, orally, topically, locally, inhalation (e.g., aerosol inhalation), injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (Remington's, 1990).

In particular embodiments, the composition is administered to a subject using a drug delivery device. Any drug delivery device is contemplated for use in delivering an effective amount of the inhibitor of mTORC1.

3. Dosage

A pharmaceutically effective amount of an inhibitor of mTORC1 is determined based on the intended goal. The quantity to be administered, both according to number of treatments and dose, depends on the subject to be treated, the state of the subject, the protection desired, and the route of administration. Precise amounts of the therapeutic agent also depend on the judgment of the practitioner and are peculiar to each individual.

The amount of rapamycin or rapamycin analog or derivative to be administered will depend upon the disease to be treated, the length of duration desired and the bioavailability profile of the implant, and the site of administration. Generally, the effective amount will be within the discretion and wisdom of the patient's physician. Guidelines for administration include dose ranges of from about 0.01 mg to about 500 mg of rapamycin or rapamycin analog.

For example, a dose of the inhibitor of mTORC1 may be about 0.0001 milligrams to about 1.0 milligram, or about 0.001 milligrams to about 0.1 milligrams, or about 0.1 milligrams to about 1.0 milligrams, or even about 10 milligrams per dose or so. Multiple doses can also be administered. In some embodiments, a dose is at least about 0.0001 milligrams. In further embodiments, a dose is at least about 0.001 milligrams. In still further embodiments, a dose is at least 0.01 milligrams. In still further embodiments, a dose is at least about 0.1 milligrams. In more particular embodiments, a dose may be at least 1.0 milligram. In even more particular embodiments, a dose may be at least 10 milligrams. In further embodiments, a dose is at least 100 milligrams or higher.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The dose can be repeated as needed as determined by those of ordinary skill in the art. Thus, in some embodiments of the methods set forth herein, a single dose is contemplated. In other embodiments, two or more doses are contemplated. Where more than one dose is administered to a subject, the time interval between doses can be any time interval as determined by those of ordinary skill in the art. For example, the time interval between doses may be about 1 hour to about 2 hours, about 2 hours to about 6 hours, about 6 hours to about 10 hours, about 10 hours to about 24 hours, about 1 day to about 2 days, about 1 week to about 2 weeks, or longer, or any time interval derivable within any of these recited ranges.

In certain embodiments, it may be desirable to provide a continuous supply of a pharmaceutical composition to the patient. This could be accomplished by catheterization, followed by continuous administration of the therapeutic agent. The administration could be intra-operative or post-operative.

4. Secondary and Combination Treatments

Certain embodiments provide for the administration or application of one or more secondary or additional forms of therapies. The type of therapy is dependent upon the type of disease that is being treated or prevented. The secondary form of therapy may be administration of one or more secondary pharmacological agents that can be applied in the treatment or prevention of vascular cognitive impairment or a disease, disorder, or condition associated with vascular pathology or vascular cognitive impairment. For example, the secondary or additional form of therapy may be directed to treating high blood pressure, high cholesterol, high blood sugar (or diabetes), an autoimmune disease, an inflammatory disease, a cardiovascular condition, or a peripheral vascular condition.

If the secondary or additional therapy is a pharmacological agent, it may be administered prior to, concurrently, or following administration of the inhibitor of mTORC1.

The interval between administration of the inhibitor of mTORC1 and the secondary or additional therapy may be any interval as determined by those of ordinary skill in the art. For example, the inhibitor of mTORC1 and the secondary or additional therapy may be administered simultaneously, or the interval between treatments may be minutes to weeks. In embodiments where the agents are separately administered, one would generally ensure that a significant period of time did not expire between the time of each delivery, such that each therapeutic agent would still be able to exert an advantageously combined effect on the subject. For example, the interval between therapeutic agents may be about 12 h to about 24 h of each other and, more preferably, within about 6 to about 12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. In some embodiments, the timing of administration of a secondary therapeutic agent is determined based on the response of the subject to the inhibitor of mTORC1.

### E. Kits

Kits are also contemplated as being used in certain aspects of the present invention. For instance, a rapamycin composition of the present invention can be included in a kit. A kit can include a container. Containers can include a bottle, a metal tube, a laminate tube, a plastic tube, a dispenser, a pressurized container, a barrier container, a package, a compartment, or other types of containers such as injection or blow-molded plastic containers into which the hydrogels are retained. The kit can include indicia on its surface. The indicia, for example, can be a word, a phrase, an abbreviation, a picture, or a symbol.

Further, the rapamycin compositions of the present invention may also be sterile, and the kits containing such compositions can be used to preserve the sterility. The compositions may be sterilized via an aseptic manufacturing process or sterilized after packaging by methods known in the art.

### EXAMPLES

The following examples are included to demonstrate certain non-limiting aspects of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention.

### Example 1

### In Vivo Effects of Rapamycin

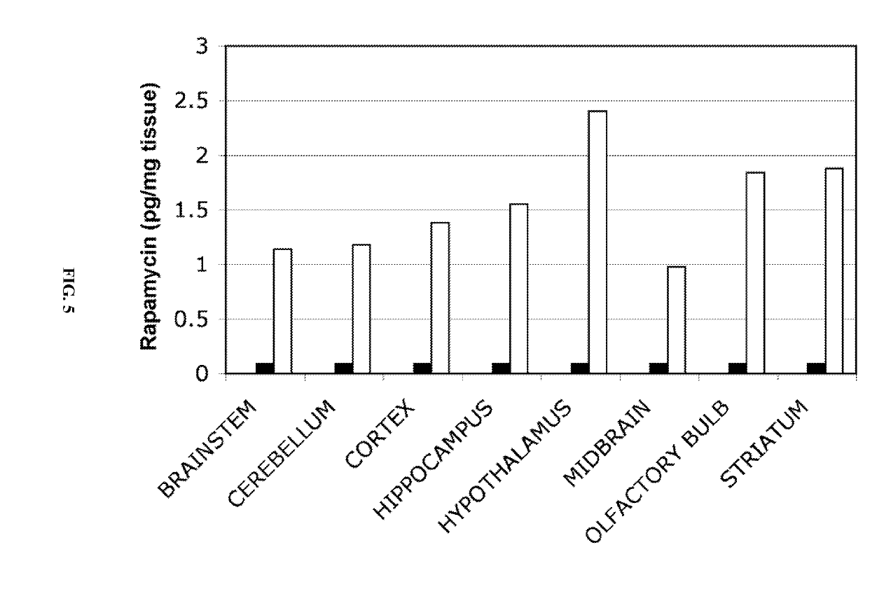

The inventors used magnetic resonance imaging (MRI) arterial spin labeling (ASL) techniques in vivo, as well as other functional imaging, in vivo optical imaging, and behavioral and biochemical tools to determine whether rapamycin treatment affects the progression of established deficits in the transgenic PDAPP mouse model of Alzheimer's Disease (Galvan, et al., 2005; Hsia, et al., 1999; Mucke, et al., 2000) (“AD mice”). AD mice and unaffected littermates were treated with rapamycin after the onset of AD-like impairments at 7 months of age (Galvan, et al., 2005; Hsia, et al., 1999; Mucke, et al., 2000) for a total of 16 weeks. Rapamycin levels in brain regions of AD mice chronically fed with rapamycin ranged from 0.98 to 2.40 pg/mg. Levels in hippocampus were 1.55 pg/mg (see FIG. 5).

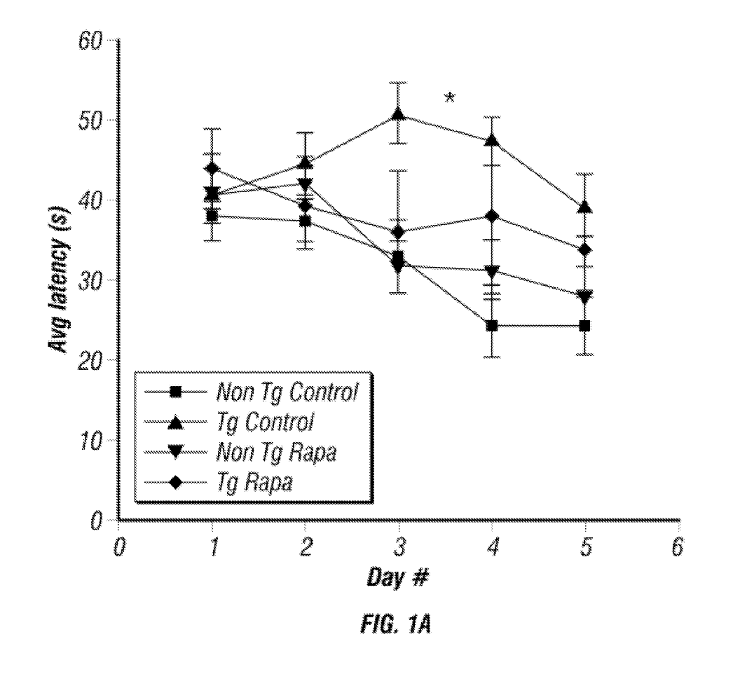

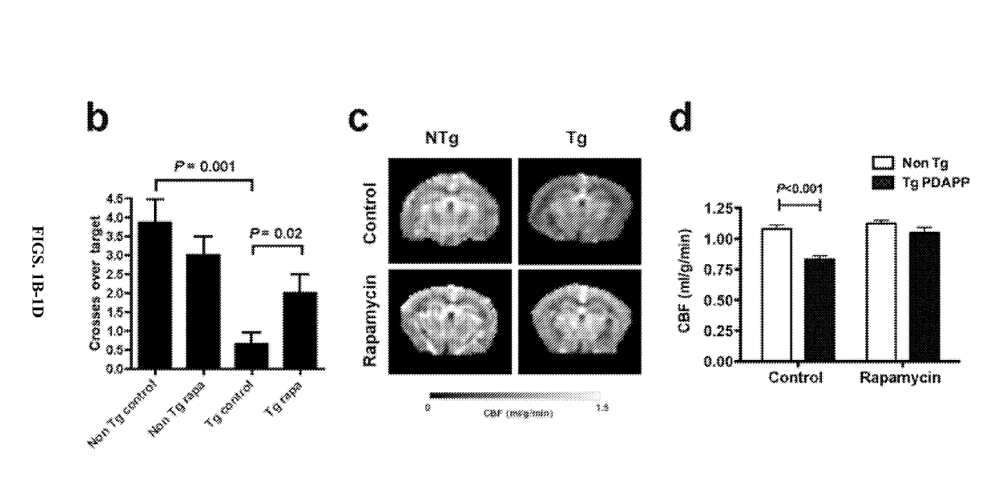

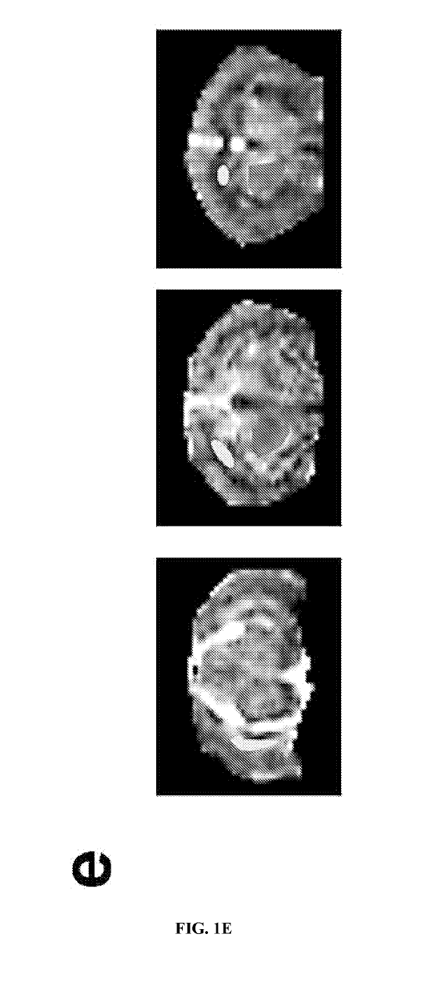

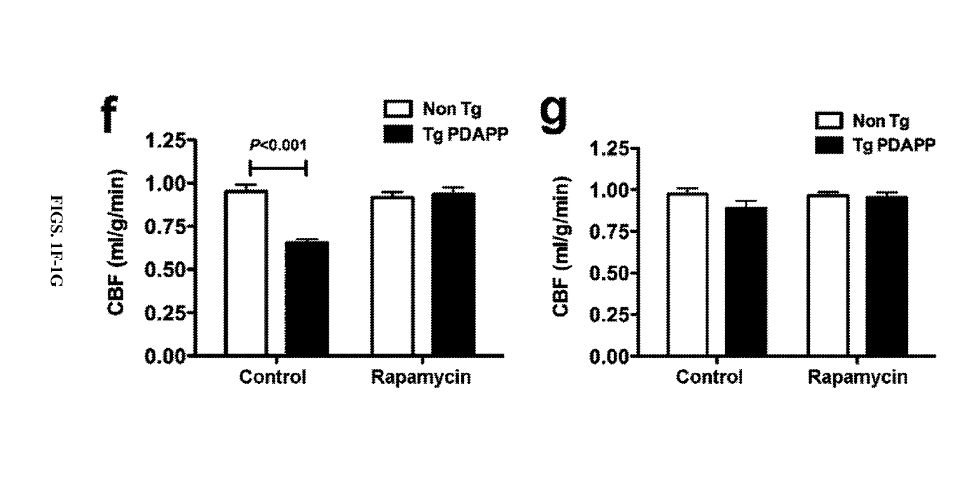

Control-fed symptomatic AD mice showed significant deficits during spatial training in the Morris water maze, as previously described (FIG. 1a) (Galvan, et al., 2005; Mucke, et al., 2000). Learning deficits of AD mice, however, were partially abrogated by rapamycin treatment. Rapamycin-induced amelioration of learning deficits was most pronounced as an inversion in the rate of acquisition early during spatial training (FIG. 1a). Control-fed AD mice showed worsening performance as training progressed, a behavioral pattern associated with increased anxiety levels in animals that do not learn well (Galvan, et al., 2008; Burger, et al., 2007; Venero, et al., 2004). In contrast, acquisition of the spatial task for the rapamycin-treated AD group improved during the first 3 days of training in a manner indistinguishable from non-transgenic littermates, but in contrast to this group, reached a plateau at day 4 (FIG. 1a). Memory of the trained location for the escape platform was significantly impaired in control-fed AD mice (FIG. 1b), as previously described (Galvan, et al., 2005; Mucke, et al., 2000; Galvan, et al., 2008). Memory in rapamycin-treated mice, however, was indistinguishable from that of non-transgenic littermates and was significantly improved compared to that of control-fed AD mice (FIG. 1b). Thus, chronic administration of rapamycin, started after the onset of AD-like cognitive deficits, improved spatial learning and restored spatial memory in symptomatic AD mice.

The inventors next examined the effects of chronic rapamycin treatment on hemodynamic function in brains of AD mice using high-field MRI (Bell & Zlokovic, 2009; de la Torre, 2004). Control-fed AD animals had significantly lower global cerebral blood flow (CBF) compared to non-transgenic littermates, (FIG. 1c-d), which indicated that the AD mice had vascular abnormalities. Global CBF in rapamycin-treated mice, in contrast, was indistinguishable from that of non-transgenic groups (FIG. 1c-d). At its earliest stages, AD is associated with synaptic dysfunction in entorhinal cortex and hippocampus while other brain regions such as thalamus are largely spared (Selkoe, et al., 2002). The inventors observed that hippocampal, but not thalamic CBF was reduced in control-treated AD mice (FIG. 1e-g). Hippocampal CBF, however, was restored to levels indistinguishable from those of non-transgenic littermates by rapamycin treatment (FIG. 1e-g).

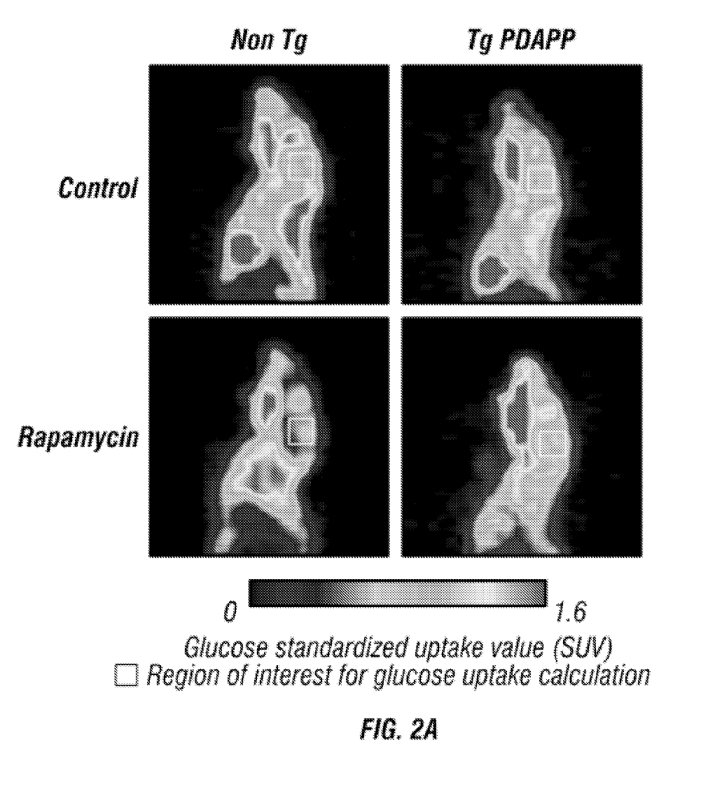

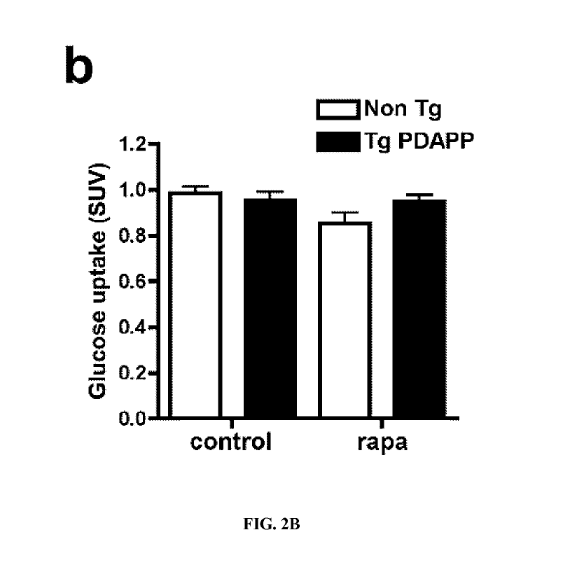

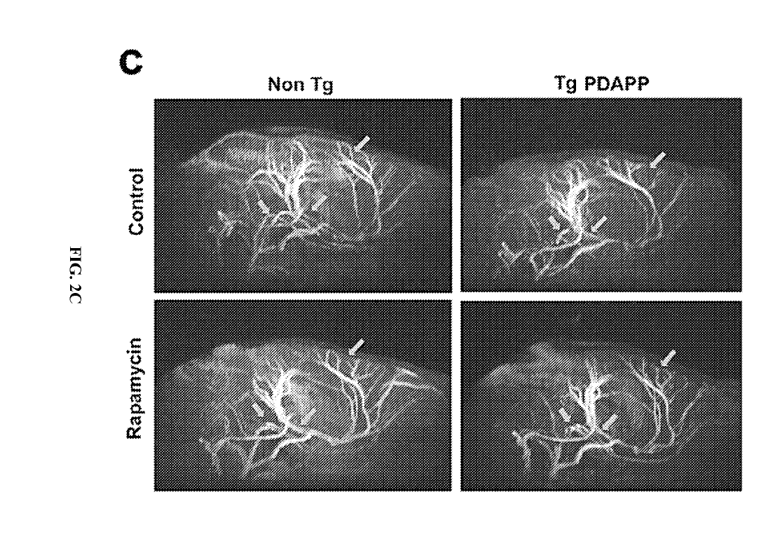

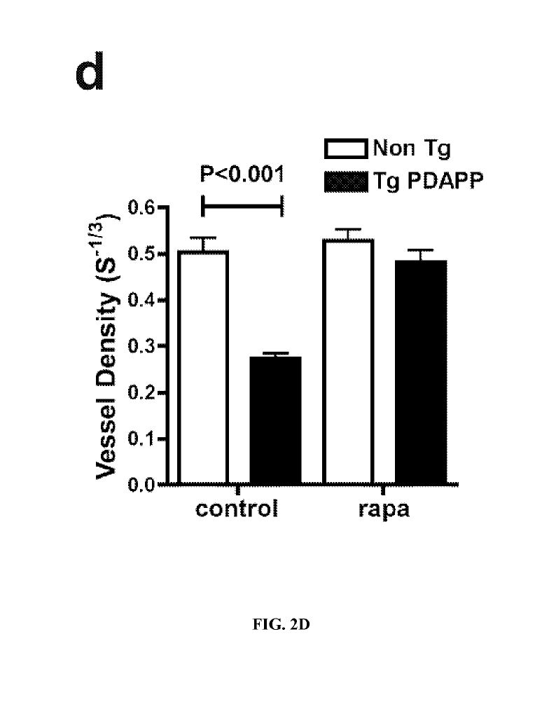

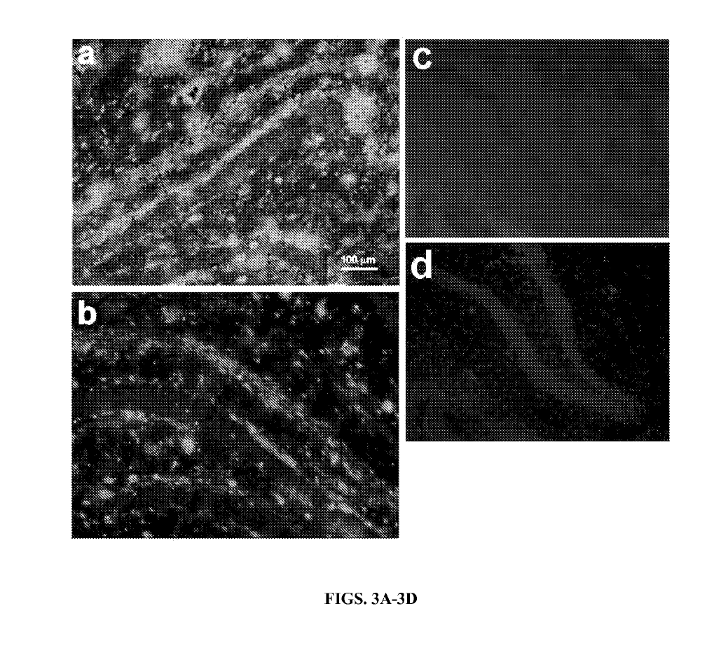

The inventors next determined cerebral glucose uptake in control- and rapamycin-fed AD mice using positron emission tomography (PET). In spite of the observed differences in CBF, cerebral metabolic rate of glucose (CMRGlc) was not significantly different between control- and rapamycin-treated groups (FIG. 2a-b). To test whether changes in CBF were caused by changes in cerebral vascularization, the inventors measured vascular density in control- and rapamycin-fed AD mouse brains using high-resolution magnetic resonance angiography (MRA). Control-treated AD mice showed a pronounced reduction in cerebral vessel density with respect to non-transgenic littermates, further demonstrating that the AD mice exhibited vascular pathology. The reduction in brain vascularity observed in the AD mice was abrogated by rapamycin treatment (FIG. 2c-d). Thus, decreases in CBF in AD mice likely arise from cerebrovascular damage, and restored CBF reflects the preservation of vascular density as a result of rapamycin treatment.

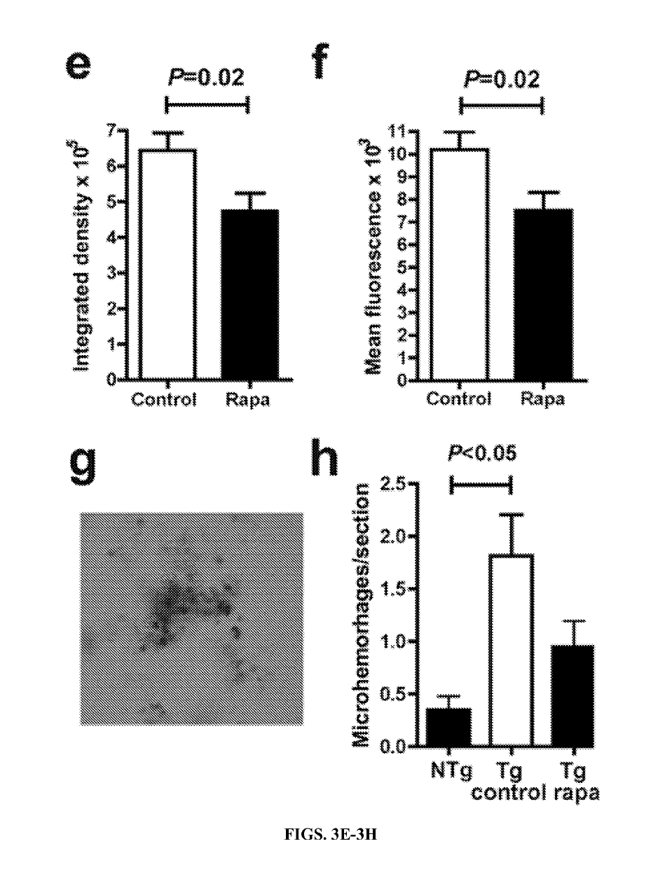

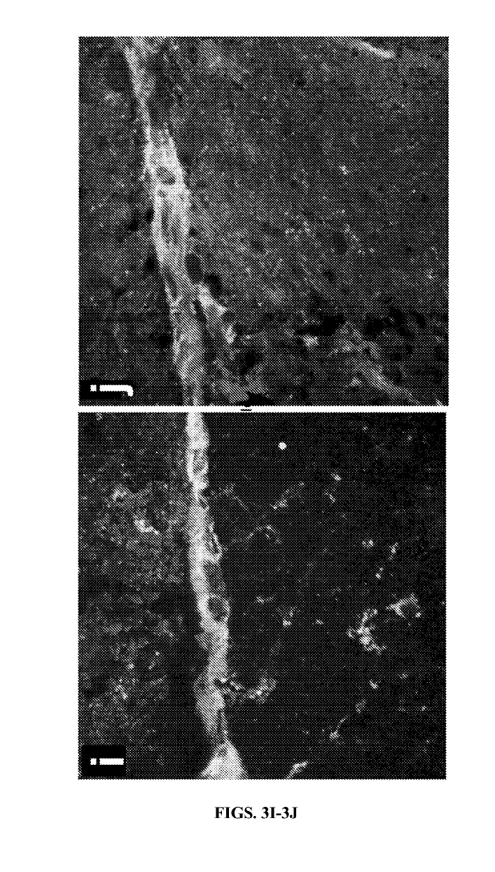

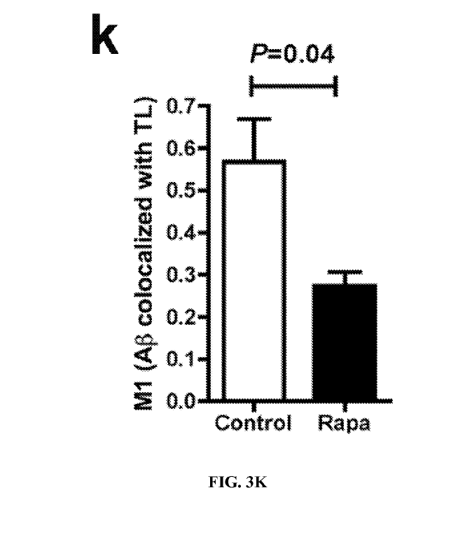

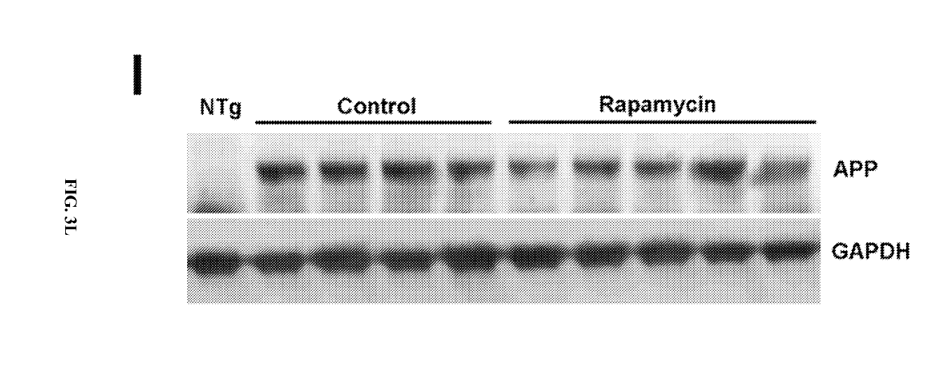

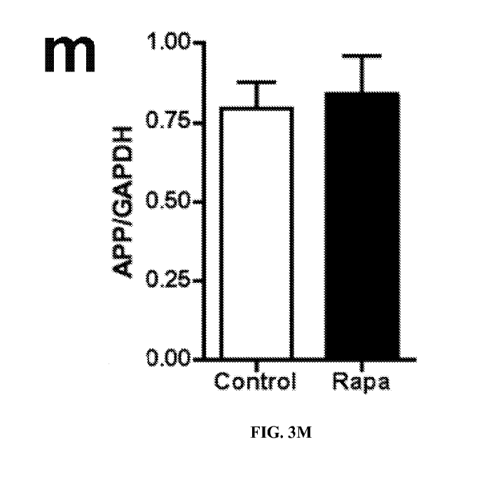

Impaired clearance of Aβ leads to its accumulation on blood vessels (Bell & Zlokovic, 2009; Sagare, et al., 2012), ultimately resulting in CAA and plaque deposition (Bell, et al., 2009). The inventors determined whether rapamycin affected Aβ plaques. Aβ deposits were significantly decreased in brains of symptomatic AD mice fed with rapamycin as compared to control-fed AD animals (FIG. 3a-f). The inventors also found that diffusivity of water was significantly increased in areas of high amyloid load as a consequence of decreased tissue integrity in control-fed AD animals, but that it was restored to normal in rapamycin-treated AD mice (FIG. 3). The inventors also quantified Aβ associated with brain blood vessels (CAA) in control- and rapamycin-treated brains. CAA was pronouncedly reduced in rapamycin-treated AD mice (FIG. 3i-k). CAA may be accompanied by microhemorrhage (Fryer, et al., 2003; Greenberg, 1998), and the inventors determined whether hemosiderin deposits, indicative of previous microhemorrhage, were present in brains of control- and rapamycin-fed animals. Hemosiderin deposits (FIG. 3g) were significantly increased in AD mouse brains (FIG. 3h) (Fryer, et al., 2003). In contrast, hemosiderin deposits in rapamycin-treated AD mice were not significantly different from those observed in non-transgenic littermates (FIG. 3h), suggesting that rapamycin-induced decreases in CAA prevented microvessel disruption in AD mouse brains. Thus, treatment of symptomatic AD mice with rapamycin decreased numbers of parenchymal plaques, and also prominently reduced vascular deposition of Aβ and microhemorrhage. Levels of transgenic human amyloid precursor protein were unchanged in control- and rapamycin-treated AD mouse brains (FIG. 31 and m), ruling out effects of rapamycin on the expression of the human amyloid precursor protein (hAPP) transgene.

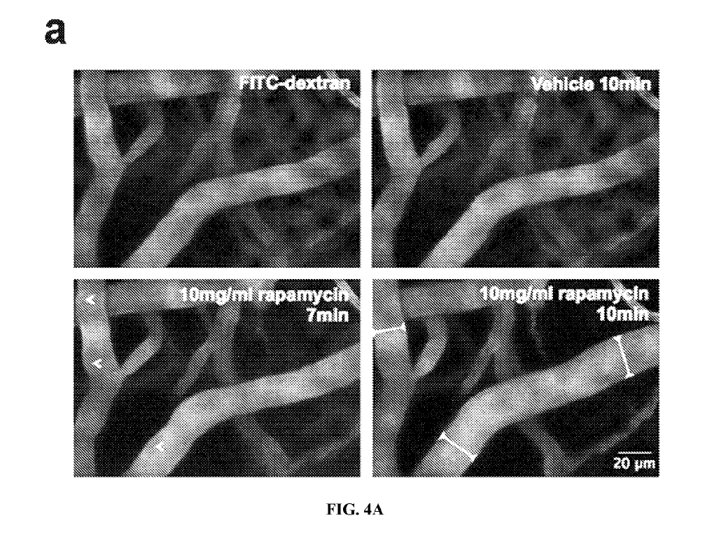

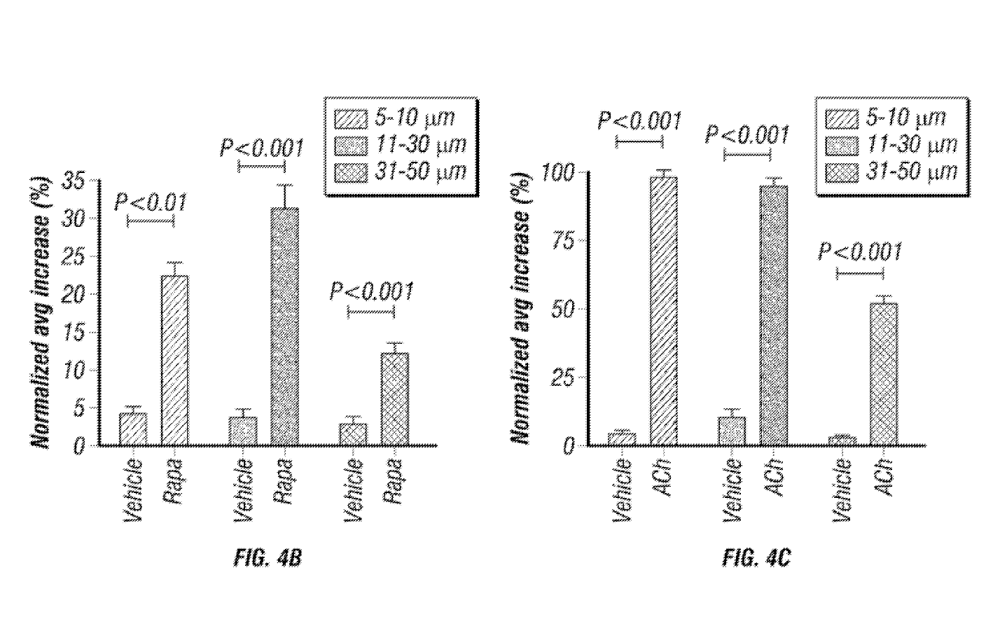

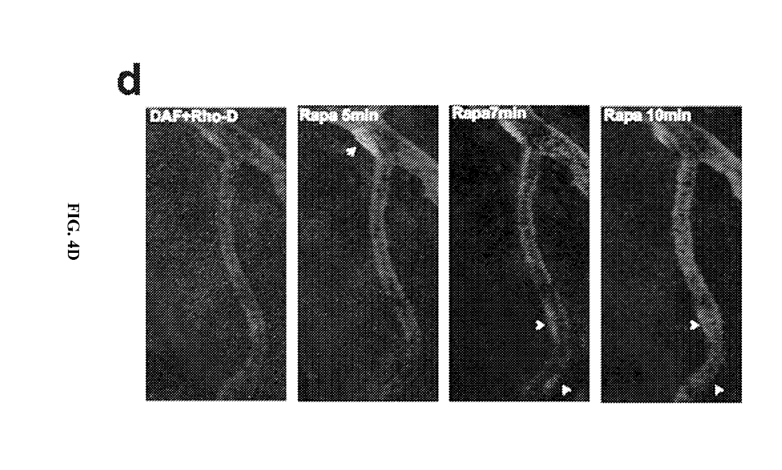

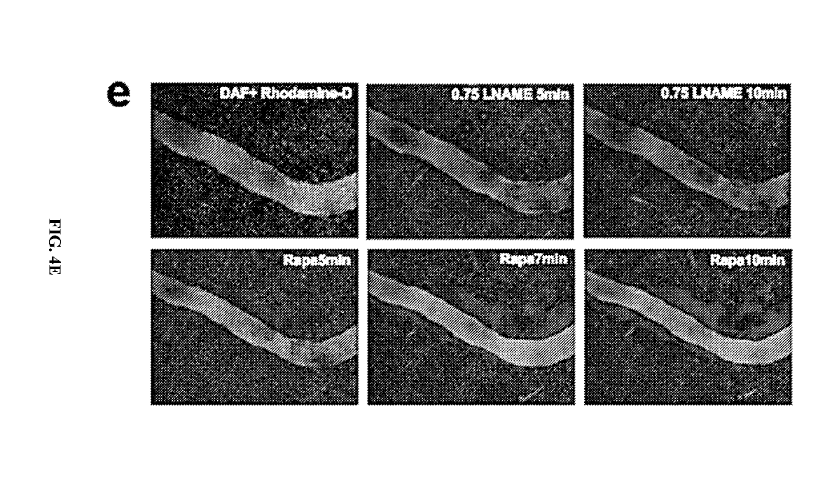

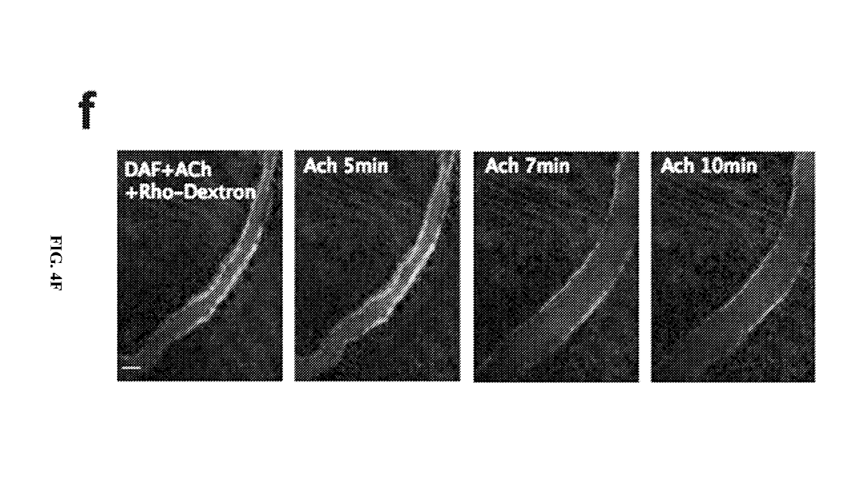

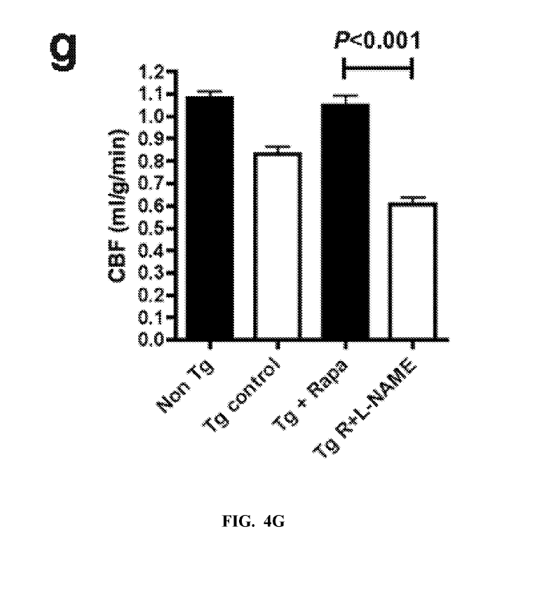

To examine the effects of rapamycin on cerebral vasculature (and thus to investigate whether rapamycin is effective against cognitive impairment that results from underlying vascular pathology), the inventors used in vivo 2-photon microscopy on cortical vessels of control- or rapamycin-treated AD mice (Bell, et al., 2009; Jellinger, 2002; Farkas & Luiten, 2001; Zlokovic, 2011). Rapamycin treatment induced a 23-35% increase in diameter of small and medium-sized cortical vessels (FIG. 4a-b). This response was roughly equivalent to one-third of the response observed after treatment with acetylcholine (ACh, FIG. 4c), a powerful vasodilator (Lee, 1982), and was comparable to that observed for other known vasodilators such as substance P (Champion & Kadowitz, 1997).

Endothelium-derived nitric oxide (NO) is an important regulator of blood flow (31). To determine whether rapamycin-induced dilation of cortical vessels was associated with NO release, the inventors used an NO-sensitive fluorescent probe to monitor NO production in cortical vessels of control- and rapamycin-treated mice. Treatment with rapamycin resulted in local increases in NO production that reached a maximum 7 minutes after treatment (FIG. 4d) and were sustained for 18 minutes. Vessel segments that showed increases in NO release subsequently increased in diameter (FIG. 4d). Treatment with ACh, on the other hand, resulted in a uniform increase in NO production along cortical vessels (FIG. 4e) that resulted in subsequent uniform increases in vessel diameter (FIG. 4e). To determine whether rapamycin-induced NO release and vasodilation were dependent on endothelial nitric oxide synthase (eNOS) activity, the inventors pretreated animals with a NOS inhibitor (L-NG-Nitroarginine methyl ester, L-NAME) before the administration of rapamycin. Pretreatment with L-NAME abrogated both NO release and vasodilation induced by rapamycin administration (FIG. 4e), indicating that eNOS-dependent NO release is required for rapamycin-induced dilation of cortical vessels.

If rapamycin-induced NO-dependent vasodilation was required for rapamycin-mediated vasoprotection (FIG. 1c-g and FIG. 2c-d), inhibition of NOS should abolish the protective effects of chronic rapamycin treatment on brain vasculature in AD mice. To test this hypothesis, the inventors treated AD mice that had been fed with rapamycin for 16 weeks starting at 7 months of age with vehicle or with L-NAME for 4 additional weeks and measured CBF in both groups. In contrast to rapamycin-fed AD animals that were injected with vehicle (FIG. 4g), rapamycin-fed mice that were injected with L-NAME showed CBF deficits comparable to control-fed AD mice, indicating that eNOS activity is required for rapamycin-dependent preservation of vascular integrity in AD mice.

The inventors' data indicate that vascular deterioration can be reversed by chronic rapamycin treatment through a mechanism that involves NO-dependent vasodilation. Rapamycin-mediated maintenance of vascular integrity led to decreased Aβ deposition in brain vessels, significantly lower Aβ plaque load, and reduced incidence of microhemorrhages in AD brains, suggesting that decreasing Aβ deposition in vasculature preserves its functionality and integrity, enabling the continuing clearance of Aβ from brain, thus resulting in decreased plaque load. Because memory deficits were ameliorated in rapamycin-treated AD mice, the inventors' data suggest that continuous Aβ clearance through preserved vasculature may be sufficient to improve cognitive outcomes in AD mice. Alternatively, a role of increased autophagy (Caccamo, et al., 2010; Spilman, et al., 2010) and the chaperone response (Caccamo, et al., 2010; Spilman, et al., 2010; Pierce, et al., 2012) may play a role.

The studies described above provide evidence for a role of mTOR in the inhibition of NO release in brain vascular endothelium during the progression of disease in AD mice, suggesting that mTOR-dependent vascular deterioration may be a critical feature of brain aging that enables AD. The inventors' data further indicate that chronic inhibition of mTOR by rapamycin, an intervention that extends lifespan in mice, negates vascular breakdown through the activation of eNOS in brain vascular endothelium, and improves cognitive function after the onset of AD-like deficits in transgenic mice modeling the disease. Rapamycin, already used in clinical settings, is expected to be an effective therapy for the vascular pathologies in AD humans and AD mice. By protecting against vascular pathologies that may cause vascular cognitive impairment, rapamycin is thus expected to be an effective therapy to prevent and treat vascular cognitive impairment.

### Example 2

### Materials and Methods

Mice. The derivation and characterization of AD [AD(J20)] mice has been described elsewhere (Hsia, et al., 1999; Mucke, et al., 2000; Roberson, et al., 2007). AD mice were maintained by heterozygous crosses with C57BL/6J mice (Jackson Laboratories, Bar Harbor, Me.). Even though the human (h)APP transgene is driven by a neuron-specific promoter that is activated at ˜e14, heterozygous crosses were set up such that the transgenic animal in was the dam or the sire in approximately 50% of the breeding pairs to avoid confounds related to potential effects of transgene expression during gametogenesis, or imprinting effects. AD mice were heterozygous with respect to the transgene. Non-transgenic littermates were used as controls. Experimental groups were: control-fed non-Tg, n=17; rapamycin-fed non-Tg, n=18; control-fed Tg, n=10; rapamycin-fed Tg, n=10, all animals were males and 11 month-old at the time of testing. Rapamycin was administered for 16 weeks starting at 7 months of age. All animal experimental protocols were approved by the Institutional Animal Care and Use Committee (IACUC) at University of Texas Health Science Center at San Antonio (Animal Welfare Assurance Number: A3345-01).

Rapamycin treatment. Mice were fed chow containing either microencapsulated enteric-coated rapamycin at 2.24 mg/kg or a control diet as described by Harrison et al., 2009. Rapamycin was used at 14 mg per kg food (verified by HPLC). On the assumption that the average mouse weighs 30 gm and consumes 5 gm of food/day, this dose supplied 2.24 mg rapamycin per kg body weight/day (Harrison, et al., 2009). All mice were given ad libitum access to rapamycin or control food and water for the duration of the experiment. Body weights and food intake were measured weekly. Food consumption remained constant and was comparable for control- and rapamycin-fed groups. Littermates (transgenic and non-transgenic mice) were housed together, thus we could not distinguish effects of genotype on food consumption. Even though there were no differences in food consumption, body weights of rapamycin-fed non-transgenic, but not transgenic, females increased moderately during treatment, (6.8% increase for rapamycin-fed vs control-fed non-transgenic females). The higher increase in body weight for non-transgenic animals is not unexpected, since non-transgenic animals of both genders tend to be slightly (1-3 g) heavier than AD transgenic.

Animal Preparation for Functional Neuroimaging. Mice were anesthetized with 4.0% isoflurane for induction, and then maintained in a 1.2% isoflurane and air mixture using a face mask. Respiration rate (90-130 bpm) and rectal temperature (37±0.5° C.) were continuously monitored. Heart rate and blood oxygen saturation level (SaO₂) were recorded using a MouseOx system (STARR Life Science Corp., Oakmont, Pa.) and maintained within normal physiological ranges.

Cerebral Metabolic Rate of Glucose (CMR_{Glc}). 0.5 mCi of ¹⁸FDG dissolved in 1 ml of physiologic saline solution was injected through the tail vein. 40 min were allowed for ¹⁸FDG uptake before scanning The animal was then moved to the scanner bed (Focus 220 MicroPET, Siemens, Nashville, USA) and placed in the prone position. Emission data was acquired for 20 min in a three-dimensional (3D) list mode with intrinsic resolution of 1.5 mm. For image reconstruction, 3D PET data was rebinned into multiple frames of is duration using a Fourier algorithm. After rebinning the data, a 3D image was reconstructed for each frame using a 2D filtered back projection algorithm. Decay and dead time corrections were applied to the reconstruction process. CMR_{Glc} was determined using the mean standardized uptake value (SUV) equation: SUV=(A×W)/Aᵢₙⱼ, where A is the activity of the region of interest (ROI; i.e., brain region in the study), W is the body weight of the mice, and Aᵢₙⱼ is the injection dose of the ¹⁸FDG(50).

Cerebral Blood Flow. Quantitative CBF (with unit of ml/min) was measured using the MRI based continuous arterial spin labeling (CASL) techniques (Duong, et al., 2000; Muir, et al., 2008) on a horizontal 7T/30 cm magnet and a 40G/cm BGA12S gradient insert (Bruker, Billerica, Mass.). A small circular surface coil (ID=1.1 cm) was placed on top of the head and a circular labeling coil (ID=0.8 cm), built into the cradle, was placed at the heart position for CASL. The two coils will be positioned parallel to each other, separated by 2 cm from center to center, and were actively decoupled. Paired images were acquired in an interleaved fashion with field of view (FOV)=12.8×12.8 mm2, matrix=128×128, slice thickness=1 mm, 9 slices, labeling duration=2100 ms, TR=3000 ms, and TE=20 ms. CASL image analysis employed codes written in Matlab (Duong, et al., 2000; Muir, et al., 2008) and STIMULATE software (University of Minnesota) to obtain CBF.

In vivo imaging experiments. Details of experimental procedures were identical to our previously published protocols (Zheng, et al., 2010). Briefly, mice were anesthetized with volatile isoflurane through a nosecone (3% induction, 1.5% maintenance). The depth of anesthesia was monitored by regular checking of whisker movement and the pinch withdrawal reflex of the hind limb and tail. Also, during surgery and imaging, three main vital signs including heart rate, respiratory rate, and oxygen saturation were periodically assessed by use of the MouseOx system (STARR Life Sciences). Body temperature was maintained at 37° C. by use of feedback-controlled heating pad (Gaymar T/Pump). Initially, the scalp was shaved, incised along the midline and retracted to expose the dorsal skull. Then removal of periosteum by forceps and cleaning of skull by a sterile cotton swab were performed. A stainless steel head plate was glutted (VetBond, 3M, St. Paul, Minn.) to dorsal skull and screwed to a custom-made stereotaxic frame. To create a thin-skull cranial window over the somotosensory cortex, skull was initially thinned by high-speed electric drill (Fine Science Tools, Foster City, Calif.) and subsequently thinned to approximate 50 μm by using a surgical blade under a dissecting microscope (Nikon SMZ800). The optimal thinness was indicated by high transparency and flexibility of skull. Artificial cerebrospinal fluid (aCSF) was used to wash the thinned area and enable pial vasculature clearly visible through the window. In vivo imaging of cortical vasculature was performed by using an Olympus FV1000 MPE with a 40× 0.8 NA water-immersion objective (Nikon). To illuminate vasculature, FITC-dextran or Rhodamine-dextran dissolved in sterilized PBS (300 μl, 10 mg/ml) was injected through tail vein at the beginning of the experiments. To observe nitric oxide (NO) derived from blood vessels, the NO indicator dye DAF-FM (Molecular Probes) was dissolved in DMSO, diluted in Rhodamine-dextran solution (250 μM), and induced into blood vessels through tail-vein injection. High-resolution z stacks of cortical layer I vasculature were sequentially acquired at different times. The NIH image J plugins stackreg and turboreg were used to align the z stacks or maximal intensity z-projections of z stacks to facilitate identification and comparison of the same blood vessels. The diameter of blood vessels was analyzed by Image J plugin vessel diameter. For the drug application, rapamycin (250 μl, 10 mg/kg solution in PBS) or a NO synthase inhibitor L-NAME (250 μl, 30 mg/kg solution in PBS) was injected intraperitoneally. Acetylcholine (300 μl, 7.5 μg/ml solution in PBS), as a positive control for vasodilation, together with Rhodamine-dextran and DAF-FM were injected intravenously via tail vein.

Behavioral testing. The Morris water maze (MWM) (54) was used to test spatial memory. All animals showed no deficiencies in swimming abilities, directional swimming or climbing onto a cued platform during pre-training and had no sensorimotor deficits as determined with a battery of neurobehavioral tasks performed prior to testing. All groups were assessed for swimming ability 2 days before testing. The procedure described by Morris et al., 1984 was followed as described (Spilman, et al., 2010; Galvan, et al., 2006; Pierce, et al., 2012). Experimenters were blind with respect to genotype and treatment. Briefly, mice were given a series of 6 trials, 1 hour apart in a light-colored tank filled with opaque water whitened by the addition of non-toxic paint at a temperature of 24.0±1.0° C. In the visible portion of the protocol, mice were trained to find a 12×12-cm submerged platform (1 cm below water surface) marked with a colored pole that served as a landmark placed in different quadrants of the pool. The animals were released at different locations in each 60′ trial. If mice did not find the platform in 60 seconds, they were gently guided to it. After remaining on the platform for 20 seconds, the animals were removed and placed in a dry cage under a warm heating lamp. Twenty minutes later, each animal was given a second trial using a different release position. This process was repeated a total of 6 times for each mouse, with each trial ˜20 minutes apart. In the non-cued part of the protocol, the water tank was surrounded by opaque dark panels with geometric designs at approximately 30 cm from the edge of the pool, to serve as distal cues. The animals were trained to find the platform with 6 swims/day for 5 days following the same procedure described above. At the end of training, a 45-second probe trial was administered in which the platform was removed from the pool. The number of times that each animal crossed the previous platform location was determined as a measure of platform location retention. During the course of testing, animals were monitored daily, and their weights were recorded weekly. Performance in all tasks was recorded by a computer-based video tracking system (Water2020, HVS Image, U.K). Animals that spent more than 70% of trial time in thigmotactic swim were removed from the study. Data were analyzed offline by using HVS Image and processed with Microsoft Excel before statistical analyses.

Western blotting and Aβ determinations. Mice were euthanized by isoflurane overdose followed by cervical dislocation. Hemibrains were flash frozen. One hemibrain was homogenized in liquid N₂ while the other was used in immunohistochemical determinations (5-7 per group). For Western blot analyses, proteins from soluble fractions of brain LN₂ homogenates were resolved by SDS/PAGE (Invitrogen, Temecula, Calif.) under reducing conditions and transferred to a PVDF membrane, which was incubated in a 5% solution of non-fat milk or in 5% BSA for 1 hour at 20° C. After overnight incubation at 4° C. with anti-APP (CT15 or anti-GFAP) the blots were washed in TBS-Tween 20 (TBS-T) (0.02% Tween 20, 100 mM Tris pH 7.5; 150 nM NaCl) for 20 minutes and incubated at room temperature with appropriate secondary antibodies. The blots were then washed 3 times for 20 minutes each in TBS-T and then incubated for 5 min with Super Signal (Pierce, Rockford, Ill.), washed again and exposed to film or imaged with a Typhoon 9200 variable mode imager (GE Healthcare, NJ). Human Aβ₄₀ and Aβ₄₂ levels, as well as endogenous mouse Aβ₄₀ levels were measured in guanidine brain homogenates using specific sandwich ELISA assays (Invitrogen, Carlsbad, Calif.) as described (Galvan, et al., 2006).

Immunohistochemistry and confocal imaging of fixed tissues. Ten-micrometer coronal cryosections from snap-frozen brains were post-fixed in 4% paraformaldehyde and stained with Aβ-specific antibodies (6E10, 10 μg/ml) followed by AlexaFluor594-conjugated donkey anti-rabbit IgG (1:500, Molecular Probes, Invitrogen, CA), and with Biotinylated Lycopersicon Esculentum (Tomato) Lectin (1:4000, Vector Laboratories, Burlingame, Calif.) followed by strepdavidin-AlexaFluor488, conjugate (1:500, Molecular Probes, Invitrogen, CA) and imaged with a laser scanning confocal microscope (Nikon Eclipse TE2000-U) using a 488 Argon laser and a 515/30 nm filter for the AlexaFluor488 fluorophore and a 543.5 Helium-neon laser and a 590/50 nm filter for the AlexaFluor594 fluorophore. Stacks of confocal images for each channel were obtained separately at z=0.15 μm using a 60× objective. Z-stacks of confocal images were processed using Volocity software (Perkin Elmer). Images were collected in the hilus of the dentate gyrus (and/or the stratum radiatum of the hippocampus immediately beneath the CA1 layer) at Bregma ˜−2.18. The MBL Mouse Brain Atlas was used for reference.

Microhemorrhages. Ten-micrometer coronal cryosections from snap-frozen brains post-fixed in 4% paraformaldehyde were washed 3× in Tris-buffered Saline (TBS) (Fisher BioReagents, NJ) and immersed in 1% Thioflavin-S (Sigma Life Sciences, St. Louis, Mo.). Sections were then washed 3× in distilled water and immersed in 2% potassium hexacyanoferrate(III) trihydrate (Santa Cruz Biotechnology, CA) and 2% hydrochloric acid (Sigma Life Sciences). After three washes in TBS, sections were coverslipped with ProLong® Gold antifade reagent with DAPI (Life Technologies, CA). The number of microhemorrhages per section was counted at Bregma ˜−2.18 using a 40× objective on a Zeiss Axiovert 200M microscope (Carl Zeiss AG, Germany) using 4 sections per animal, and numbers of microhemorrhages were averaged for each animal.

Statistical analyses. Statistical analyses were performed using GraphPad Prism (GraphPad, San Diego, Calif.) and StatView. In two-variable experiments, two-way ANOVA followed by Bonferroni's post-hoc tests were used to evaluate the significance of differences between group means. When analyzing one-variable experiments with more than 2 groups, significance of differences among means was evaluated using one-way ANOVA followed by Tukey's post-hoc test. Evaluation of differences between two groups was evaluated using Student's t test. Values of P<0.05 were considered significant.

### Example 3

### Other Animal Models of Vascular Cognitive Impairment

Other animal models of vascular cognitive impairment (including rodent models) may be used to further characterize the beneficial effects of rapamycin treatment that were observed in the studies described above (Nishio, et al., 2010; Ihara & Tomimoto, 2011; Tomimoto, 2005). Such rodent models may be tested as described above in Examples 1 and 2. For example, rodent subjects may be administered rapamycin or a negative control and subsequently evaluated using the behavioral, imaging, biochemical, and metabolic and blood flow protocols described in Examples 1 and 2.

### REFERENCES

The following references, to the extent that they provide exemplary procedural or other details supplementary to those set forth herein, are specifically incorporated herein by reference.
Atochin et al., J Clin Invest. 117:1961, 2007.
Bell et al., Nat Cell Biol. 11:143, 2009.
Bell & Zlokovic, Acta Neuropathol. 118:103, 2009.
Bird, J Endocrinol. 210(3):239-41, 2011.
Breteler, Ann NY Acad Sci. 903:457, 2000.
Burger et al., Neurobiol Learn Mem. 87:21, 2007.
Caccamo, et al., J Biol Chem. 285:13107, 2010.
Champion & Kadowitz, Am J Physiol. 273:R414, 1997.
Cheng et al., Cardiovasc Res 78:123, 2008.
Chin et al., J Neurosci. 27:2727, 2007.
de la Torre, Lancet Neurol. 3:184, 2004.
Duong, Magn Reson Med. 43:383, 2000.
Farkas & Luiten, Prog Neurobiol. 64:575, 2001.
Fryer et al., J Neurosci. 23:7889, 2003.
Galvan et al., Behav Brain Res. 191:246, 2008.
Galvan et al., Proc Natl Acad Sci USA. 103:7130, 2006.
Greenberg, Neurology. 51:690, 1998.
Harrison et al., Nature. 460:392, 2009.
Hsia et al., Proc Natl Acad Sci USA. 96:3228, 1999.
Iadecola, Nat Rev Neurosci. 5:347, 2004.
Ihara & Tomimoto, J of Aging Research. 2011:978761, 2011.
Jellinger, J Neural Transm. 109:813, 2002.
Koide et al., Biol Pharm Bull. 34:602, 2011.
Lee et al., J Biomed Biotechnol. 2011:535902, 2011.
Lee, Circ Res. 50:870, 1982.
Marin & Rodriguez-Martinez, Exp Gerontol. 34:503, 1999.
McGuane et al., Hypertension. 57:1151, 2011.
Meilandt et al., J Neurosci. 29:1977, 2009).
Miller et al., J Gerontol A Biol Sci Med Sci. 66:191, 2011.
Morris, J Neurosci Methods. 11:47, 1984.
Mucke et al., J Neurosci. 20:4050, 2000.
Muir, et al., Magn Reson Med. 60:744, 2008.
Naoum et al., Surgery. 136:323, 2004.
Nishio, et al., Stroke. 41(6):1278-84, 2010
Owen et al., Free Radic Biol Med. 49:1798, 2010.
Petrovitch et al., Ann Neurol 57, 98, 2005.
Pierce et al., J Neurochem, 2012.
Roberson et al., Science. 316:750, 2007.
Rubbo et al., Arch Biochem Biophys. 324:15, 1995.
Sagare, et al., J Alzheimers Dis. 2012.
Sarbassov, et al., Curr Opin Cell Biol. 17:596, 2005.
Schneider, et al., Ann Neurol. 62:59, 2007.
Selkoe, Science. 298:789, 2002.
Shah, et al., Curr Biol. 14:1650, 2004.
Shaw & Cantley, Nature. 441:424, 2006.
Shibata et al., J Clin Invest. 106:1489, 2000.
Snowdon et al., JAMA. 277:813, 1997.
Spilman et al., PLoS One. 5:e9979, 2010.
Sun et al., Cancer Res. 65:7052, 2005.
Thal et al., Neurobiol Aging. 30:1936, 2009.
Tomimoto, et al., J. of Cerebral Blood Flow & Metabolism. 25:S263, 2005.
Tremblay, et al., Endocrinology. 14:1328, 2005.
Venero et al., J Neurosci Res. 75:172, 2004.
Zachary, Am J Physiol Cell Physiol. 280:C1375, 2001.
Zheng et al., PLoS One. 5:e14401, 2010.
Zlokovic, Nat Rev Neurosci. 12:723, 2011.

1. A method for treating or preventing vascular cognitive impairment, the method comprising administering an effective amount of a composition comprising rapamycin or an analog of rapamycin to a subject having or suspected of having vascular cognitive impairment.

2. The method of claim 1, wherein the subject has been diagnosed as having vascular cognitive impairment.

3. The method of claim 1, wherein the composition comprising rapamycin or an analog of rapamycin is orally administered to the subject.

4. The method of claim 1, wherein the composition comprising rapamycin or an analog of rapamycin is a nanoparticle formulation.

5. The method of claim 1, wherein the composition comprising rapamycin or an analog of rapamycin further comprises a hydrophilic, swellable, hydrogel forming material.

6. The method of claim 1, wherein the composition comprising rapamycin or an analog of rapamycin further comprises a thermoplastic polymer.

7. The method of claim 1, wherein the subject is administered a composition comprising rapamycin or an analog of rapamycin and a composition comprising a second active agent.

8. The method claim 7, wherein the second active agent comprises an agent that increases eNO, a stimulator of eNOS, a cholinesterase inhibitor, an anti-glutamate, an anti-hypertensive agent, an anti-platelet agent, an antihyperlipidemic agent, or a medication that alleviates or treats low blood pressure, cardiac arrhythmia, or diabetes.

9. The method of claim 8, further comprises an agent that increases the stability of eNOS.

10. The method of claim 8, wherein the cholinesterase inhibitor is tacrine, donepezil, rivastigmine, or galantamine or analogs thereof.

11. The method of claim 8, wherein the anti-glutamate is memantine or analogs thereof.

12-14. (canceled)

15. The method of claim 1, wherein the composition comprising rapamycin or an analog of rapamycin is encased in a coating that includes a water insoluble polymer and a hydrophilic water permeable agent.

16. The method of claim 15, wherein the water insoluble polymer is a methyl methacrylate-methacrylic acid copolymer.

17. The method of claim 1, wherein the subject is a human, dog, or cat.

18. The method of claim 17, wherein the subject is a human.

19-20. (canceled)

21. The method of claim 1, wherein the subject has high blood pressure, high cholesterol, high blood sugar, diabetes, an autoimmune disease, or an inflammatory disease.

22-24. (canceled)

25. The method of claim 18, wherein the human subject is greater than age 50.

26-29. (canceled)

30. The method of claim 1, wherein the composition comprising rapamycin or an analog of rapamycin comprises 25% to 60% by weight of rapamycin or an analog of rapamycin.

31. The method of claim 1, wherein the 24 hour trough level of rapamycin or an analog of rapamycin is greater than 1 ng/ml whole blood after administration of the composition.

32. The method of claim 1, wherein the average tissue level of rapamycin in the subject is greater than 0.75 pg per mg of tissue after administration of the composition.

33-58. (canceled)
